(19) United States
(12) Patent Application Publication
(10) Pub. No.: US 2026/0069227 A1
(43) Pub. Date: Mar. 12, 2026

(54) TARGET VESSEL RECONSTRUCTION FOR NON-RIGID MOTION COMPENSATION

(71) Applicant: Siemens Healthineers AG, Forchheim (DE)

(72) Inventors: Alexander Preuhs, Erlangen (DE); Jens-Christoph Georgi, Oberasbach (DE)

(21) Appl. No.: 19/324,538

(22) Filed: Sep. 10, 2025

(30) Foreign Application Priority Data
Sep. 12, 2024 (DE) 10 2024 208 695.9

(51) Int. Cl. A61B 6/50; A61B 6/00; G06T 5/77; G06T 5/94; G06T 15/08

(52) U.S. Cl. CPC A61B 6/504; A61B 6/481; A61B 6/5205; A61B 6/5264; G06T 5/77; G06T 5/94; G06T 15/08; G06T 2207/30101; G06T 2210/41

(57) ABSTRACT

A method for motion-compensated reconstruction of a target vessel by providing a primary 3D reconstruction of a volume including the target vessel, identifying an image of the target vessel and an image of a secondary vessel different from the target vessel in the primary 3D reconstruction, forward-projecting the image of the target vessel and the image of the secondary vessel onto forward projection images, erasing the image of the secondary vessel in the forward projection images, and generating a secondary 3D reconstruction from the forward projection images with automatic motion compensation.

### CROSS REFERENCE TO RELATED APPLICATIONS

This application claims the benefit of DE 10 2024 208 695.9 filed on Sep. 12, 2024, which is hereby incorporated by reference in its entirety.

### FIELD

Embodiments relate to a method for motion-compensated reconstruction of a target vessel and to an image processing apparatus for motion-compensated reconstruction as well as to a computed tomography apparatus, a computer program, and a computer-readable storage medium.

### BACKGROUND

In interventional oncology, for example in tumor embolization, the blood vessels supplying the cancer tissue have to be blocked to trigger tumor necrosis. Moreover, as little healthy tissue as possible should be affected by the blockade, to avoid necrosis of the healthy tissue. Contrast-enhanced 3D reconstructions may be used for this purpose. The 3D reconstruction makes it possible to identify the particular vessels that need to be blocked, i.e., the “feeder vessels”(inflow vessels).

To achieve the above-stated effects, the 3D reconstruction of the contrast-enhanced vessels must be as accurate as possible. Non-rigid motion artifacts in the organs, for example due to motion artifacts, are a significant obstacle to high quality vessel reconstruction. While rigid motion for example moves the entire organ, non-rigid motion takes place within the organ itself, i.e., is location-dependent.

Using contrast-enhanced angiographic images to achieve high-precision motion compensation for vascular supply is desirable.

One common solution are motion compensation reconstructions, as presented in the article by Rohkohl, C., Lauritsch, G., Biller, L., Prümmer, M., Boese, J., & Hornegger, J. (2010); Interventional 4D motion estimation and reconstruction of cardiac vasculature without motion periodicity assumption, Medical image analysis, 14(5), 687-694.

Due to the complexity of the 3D imaging field, however, these algorithms often fail in the case of relatively small vascular structures.

In general, approaches to motion compensation attempt to maximize an image optimization criterion based on a) the projection space, b) the reconstruction space, or c) a combination of the two. A problem that frequently arises with corresponding optimization algorithms is that there are different optimum solutions for different anatomical regions. For example, osseous structures may not move significantly, while soft tissue structures may move considerably. Motion compensation may then lead to blurring of the osseous structures, or indeed the soft tissue regions may be poorly compensated because the optimization algorithm is concentrating on the osseous regions. One familiar approach to mitigating these problems is to define a target volume, or the distance from bone, in which optimization is to be carried out.

The article by Unberath et al. (2016) entitled Virtual single-frame subtraction imaging, in Proc. Conf. Imag. Form. X-Ray CT (pp.89-92) outlines a general framework for single-frame detector-domain material decomposition. The method involves a segmentation step and a background estimation step yielding a virtual mask image that may be used for subtraction. In many cases, material decomposition yields non-truncated difference images enabling the use of novel motion estimation methods that exploit epipolar consistency conditions.

Furthermore, the article by Preuhs, A., Manhart, M., Roser, P., Stimpel, B., Syben, C., Psychogios, M., . . . & Maier, A; (2020) entitled Deep autofocus with cone-beam CT consistency constraint, in Bildverarbeitung für die Medizin 2020: Algorithmen-Systeme-Anwendungen [Image processing for medicine 2020: Algorithms-systems-application], Proceedings of the workshop on March 15-17, 2020 in Berlin (pp. 169-174), Springer Fachmedien Wiesbaden, proposed a novel learning-based approach that is capable of compensating motion within the acquisition plane. The paper introduces a CBCT consistency constraint that has proven to be reliable for motion perpendicular to the acquisition plane. This allows good detection of motion within and outside the plane, achieving average artifact suppression of 93%.

According to the article by Preuhs, A., Berger, M., Bauer, S., Redel, T., Unberath, M., Achenbach, S., & Maier, A. (2018) entitled Viewpoint planning for quantitative coronary angiography, International Journal of Computer Assisted Radiology and Surgery, 13, 1159-1167, in coronary angiography, the condition of myocardial blood supply is assessed by analyzing 2D X-ray projections of contrasted coronary arteries. This takes place using a flexible C-arm system. Due to the X-ray immanent dimensionality reduction when projecting the 3D scene onto a 2D image, the viewpoint is critical when it comes to guaranteeing an appropriate view onto the affected artery and thus enabling reliable diagnosis. To this end, an algorithm is presented that computes optimal viewpoints for the assessment of coronary arteries without the need for 3D models. Optimal viewpoint planning is performed solely on the basis of a single angiographic X-ray image.

### BRIEF SUMMARY AND DESCRIPTION

The scope of the embodiments is defined solely by the appended claims and is not affected to any degree by the statements within this summary. The present embodiments may obviate one or more of the drawbacks or limitations in the related art. Independent of the grammatical term usage, individuals with male, female or other gender identities are included within the term.

Embodiments improve the reconstruction of a target vessel that is subject to non-rigid motion.

According to an embodiment, a method is provided for motion-compensated reconstruction of a target vessel. The target vessel is for example a “feeder vessel” with which a tumor is supplied with blood. However, the target vessel may also be a bile duct or the like. The intention is to reconstruct this target vessel without motion artifacts.

To this end, firstly a primary 3D reconstruction of a volume including the target vessel is provided. The term “provided” may for example be understood to mean received, read out, acquired etc., for example acquired and/or read out from a computer-readable data memory and/or received from a data storage unit, for example a database. The provision serves to make the data from the primary 3D reconstruction available to the method. The target vessel is thus spatially represented in the primary 3D reconstruction. This primary 3D reconstruction is typically disrupted by motion artifacts. It would, for example, be difficult to use this disrupted 3D reconstruction to introduce a catheter into the target vessel.

Therefore, in a further step, an image of the target vessel and an image of a second vessel different from the target vessel are identified in the primary 3D reconstruction (below, the terms “target vessel” and “secondary vessel” are understood respectively to mean an “image of the target vessel” and an “image of the secondary vessel”, respectively, unless otherwise explicitly stated or unless the context suggests otherwise). In addition to the secondary vessel, many further secondary vessels may of course be present in the primary 3D reconstruction. As a rule, all these secondary vessels disrupt automatic motion compensation algorithms. The aim is therefore to remove as many secondary vessels from the volume of the primary 3D reconstruction as possible, in order to be able to focus motion compensation on the target vessel. To this end, the target vessel is identified, for example manually by labeling or automatically for example by image processing. Identification of the image of the target vessel and of the image of the secondary vessel different from the target vessel may involve annotation and/or segmentation, for example including threshold values and/or time-intensity curves, of respective image dots of the primary 3D reconstruction. The image dots of the primary 3D reconstruction may, for example, take the form of pixels and/or voxels and have respective image values that represent the volume including the target vessel and the secondary vessel.

In a further step, the target vessel and the secondary vessel are then forward-projected onto forward projection images. Forward projection may image, for example map, for example virtually, a 3D object onto a two-dimensional plane. Different forward projection images of the 3D object may be obtained depending on the projection direction of the forward projection. Forward projection images are 2D images in a projection image space. As a rule, all the other secondary vessels are also forward-projected with the secondary vessel. The purpose of this forward projection is that it is easier to erase the secondary vessels in the two-dimensional projection image space than in the 3D reconstruction. The expression “erasing the secondary vessel” should be understood to mean that the influence of the secondary vessel in the respective image is reduced to below a predetermined limit and optionally minimized. This also means, however, that the influence of other organs, for example, on the respective pixels is not generally reduced.

The secondary vessel is therefore erased in the forward projection images in a further step. It goes without saying that, here too, as far as possible all other secondary vessels may be erased. Erasure is performed, for example, by reducing the contrast or by overpainting the secondary vessel or secondary vessels. The secondary vessel does not therefore have to be completely erased, but should be for the most part. For example, the contrast between the secondary vessel and its surroundings should be reduced to at least a quarter.

The forward projection images therefore no longer contain the secondary vessels or at most contain them as shadows. In a subsequent step, a secondary 3D reconstruction is generated from the forward projection images with automatic motion compensation. The secondary 3D reconstruction is thus reconstructed from the two-dimensional forward projection images without or for the most part without secondary vessels. The 3D reconstruction involves automatic motion compensation, for which the compensation algorithm is able to concentrate on the target vessel. Optimal motion compensation in terms of the target vessel is accordingly possible. The resultant secondary 3D reconstruction therefore depicts the target vessel with motion artifacts that are generally greatly reduced compared to the primary 3D reconstruction.

One embodiment provides that motion blur of the secondary vessel arises in the respective forward projection image during forward projection, the motion blur being taken into account during erasure with a spatial safety region, for example a safety margin, around the image of the secondary vessel in the forward projection images. Since the secondary vessel may be blurred in the primary 3D reconstruction due to motion, the secondary vessel is accordingly also blurred in the two-dimensional forward projection. This blur should be taken into account during erasure of the secondary vessel. It is therefore advantageous for the spatial safety region, i.e., for example a predetermined region, around the center line of a vessel to be erased during erasure of the spatial safety region. Erasure may be performed manually or automatically for example with a corresponding algorithm. This spatial safety region ensures that the largest possible proportion of pixels of the secondary vessel is erased when a typical motion range of the secondary vessel is taken into account.

In one embodiment, an inpainting algorithm is used to erase the secondary vessel. In this case, the secondary vessel or a region around the secondary vessel is overpainted. The inpainting algorithm is thus not used here to fill a gap, but rather deliberately to draw over an identified vessel region. In this case, the region to be infilled (here the secondary vessel region) is typically filled in with pixels that are adapted to the surroundings of the secondary vessel region in terms of brightness. In this way, the secondary vessel region including the secondary vessel would be completely erased, unless not all the pixels of the secondary vessel region had been filled in using the inpainting method.

According to an embodiment, the secondary vessel is erased in that two projection images in the same motion state but at different projection angles are provided for each of the forward projection images, vascular structures of the secondary vessel being extracted from these, wherein the secondary vessel is erased in the respective forward projection image if the extracted vascular structures and the secondary vessel in the respective projection image fulfill a consistency condition. Vascular structures may thus be iteratively erased (for example, by overpainting (inpainting) or (complete) contrast reduction), if one or more consistency conditions are fulfilled in different motion phases in respect of vascular structures of the secondary vessel obtained from other projection images. In one specific case, at least two projection images with the same motion state and at least 20 degrees of projection angle difference (cf. Preuhs, A. et al. (2018)) may be obtained for each image. The two images may be used for iterative erasure of the vascular structures that are not included in the projection by extracting them from a new projection image. The aim of optimization is to maximize the consistency of the newly produced projection images (i.e., of the extracted vessels). This makes it possible, as with the inpainting method, to obtain a set of projection images without secondary vessels, for example without images of secondary vessels. In the case of tumor embolization, the set of projection images thus no longer contains any non-feeder vessels, for example no images of non-feeder vessels.

A further embodiment provides that non-rigid motion is compensated during motion compensation. This means for example that location-dependent motion may be compensated within the 3D reconstruction space. The motion compensation is thus capable of eliminating or compensating complex motion fields.

According to a further embodiment, the target vessel is an outflow vessel or inflow vessel for a given region of an object, and the secondary vessel is accordingly a non-outflow vessel or non-inflow vessel for the given region. For example, the target vessel is an inflow vessel (feeder vessel) to a tumor, while the secondary vessel does not provide inflow to the tumor. In the case of a secretory organ, the target vessel may be an outflow vessel, with the secondary vessel being a non-outflow vessel. For example, the outflow vessels of the liver and pancreas drain into a common main duct that leads into the small intestine. The pancreatic duct from the pancreas would in this case constitute a non-outflow vessel of the liver.

Such distinctions between inflow and non-inflow vessel or outflow and non-outflow vessel may in principle be drawn at each vessel branching point, since at such points inflow and outflow always take place respectively to and from different directions or regions.

The above object is also achieved by an image processing apparatus for motion-compensated reconstruction of a target vessel having a storage device for providing a primary 3D reconstruction of a volume including the target vessel, and computing device that is configured to identify the target vessel and a secondary vessel different from the target vessel in the primary 3D reconstruction, wherein the target vessel has a higher contrast (relative to its surroundings) than the secondary vessel, forward-project the target vessel and the secondary vessel onto forward projection images, erase (reduce the contrast of) the secondary vessel in the forward projection images, and generate a secondary 3D reconstruction from the forward projection images (without the secondary vessels) with automatic motion compensation.

The storage device may have one or more storage elements or modules.

Optionally it also has its own processor. The storage device may be implemented locally or indeed also in a distributed manner on the internet.

The computing device may be implemented by a computer. As a rule, it has one or more processors and optionally its own storage elements. Furthermore, it typically has input and output interfaces.

The advantages and variant embodiments outlined above in relation to the method are also applicable mutatis mutandis to the image processing apparatus. Accordingly, the stated method features form functional features of the image processing apparatus.

Advantageously, a computed tomography apparatus (CT apparatus) may also be provided with such an image processing apparatus. The image processing apparatus processes signals that are obtained from a detector of the computed tomography apparatus.

The advantages and variant embodiments outlined above in relation to the method and/or the image processing apparatus are also applicable mutatis mutandis to the computed tomography apparatus. Accordingly, the stated method features form functional features of the computed tomography apparatus.

Furthermore, a computer program may also be provided that includes commands that, when the program is executed by the above image processing apparatus, cause the latter to execute the above-described method. Similarly, a computer storage medium may be provided that includes commands that, when the program is executed by the above image processing apparatus, cause the latter to execute the above-described method.

### BRIEF DESCRIPTION OF THE FIGURES

FIG. 1 depicts a schematic representation of a computed tomography apparatus according to an embodiment.

FIG. 2 depicts a sequence diagram of an embodiment of a method.

FIG. 3 depicts a tomographic image from the 3D reconstruction with identified target vessel according to an embodiment.

FIG. 4 depicts a forward projection based on the 3D volume set from FIG. 3 with erased secondary vessels according to an embodiment.

### SUMMARY AND DESCRIPTION

Cone-beam computed tomographs (CBCT) and flat panel detector angiography systems are frequently used for interventional purposes, for example for intraoperative tracking of instruments or guidance of such instruments.

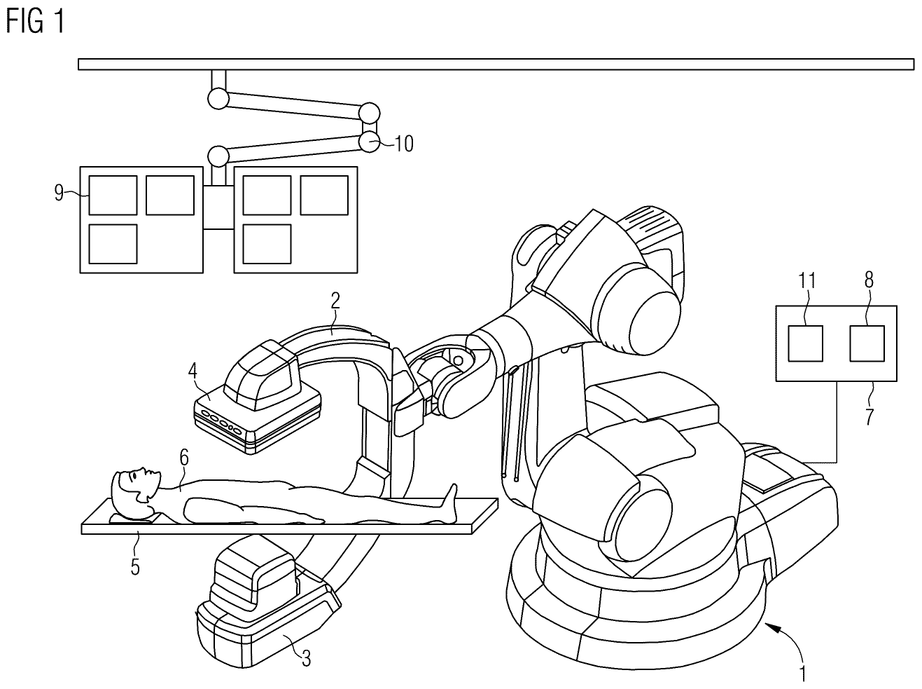

FIG. 1 depicts, by way of example for a computed tomography apparatus, a schematic representation of a monoplanar X-ray system with a C-arm 2 supported by a stand 1 in the form of a six-axis industrial or buckling-arm robot, at the end of which C-arm an X-ray source, for example an X-ray emitter 3 with X-ray tube and collimator, and an X-ray image detector 4 are mounted as an image capture unit. Implementation of the X-ray diagnostic apparatus is not dependent on the industrial robot. Conventional C-arm equipment may also be used.

A patient 6 to be examined or a technical object is located as object under examination on a table top 5 of a patient positioning table in the beam path of the X-ray emitter 3. A system control unit 7 with a computing device 8 for image processing, that receives and processes the image signals from the X-ray image detector 4, is connected to the X-ray diagnostic device (operator control elements, for example, are not shown). The X-ray images may then be observed on displays on a monitor rack 9. The monitor rack 9 may be supported by way of a ceiling-mounted, longitudinally displaceable, swivelable, rotatable and height-adjustable support system 10 with bracket and lowerable carrier arm. The system control unit 7 may furthermore have a storage device 11 for the computing device 8, in order, for example, to provide 3D reconstructions and/or 2D projection images.

A 3D reconstruction of a region of interest of the patient 6 (or in general of an object) may be obtained from two-dimensional projection images. Comprehensive motion compensation is usually required. In one embodiment, the reconstruction space and the image space are combined to this end by enforcing consistency between the two spaces. To this end, the two spaces are transformed into a common space, for example, by back-projection of the projection images into the reconstruction space or vice versa. A motion field may then be optimized using a quality metric. Ideally, the motion field reflects the true, non-rigid patient movement when the quality metric is convergent.

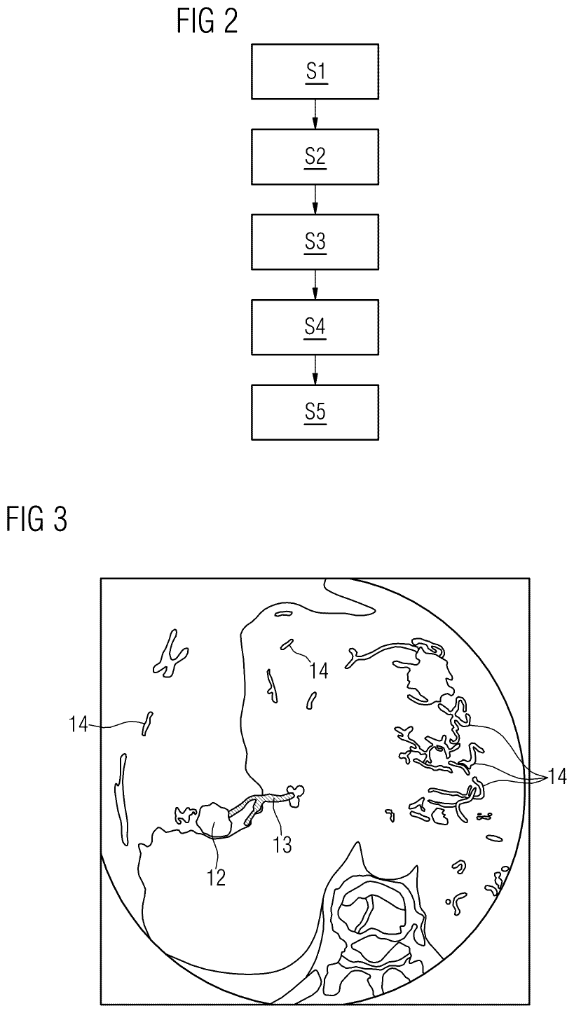

An improved strategy for motion compensation is proposed, according to which the target volume is restricted to a clinically relevant minimum and the results of motion compensation are thus improved. The input for this method is for example a stack of projection images with contrast-enhanced vascular structures. On the basis of this input, the proposed method may be subdivided into five steps, that are described hereinafter with reference to FIG. 2.

In a first step S1, a primary 3D reconstruction of a volume including the target vessel is provided, for example reconstructed from the stack of projection images. The primary 3D reconstruction is optionally also directly provided; reconstruction having taken place at an earlier point in time.

In a second step S2, the target vessel and (at least) one secondary vessel different from the target vessel are identified in the primary 3D reconstruction. For example, the supply vessels in the 3D space (i.e., the vessels of interest) may thus be identified. The 3D vascular tree is then subdivided into feeder and non-feeder vessels.

In a third step S3, the target vessel and the secondary vessel are forward-projected onto forward projection images in the two-dimensional projection image space. The non-feeder vessels and feeder vessels in the projection image space are optionally identified again. In the case of moving vessels, the respective forward projection takes account of motion blur. A certain spatial safety region should therefore being taken into account for the forward projection.

In a fourth step S4, the secondary vessel is erased (for example, by overpainting (inpainting) or complete contrast reduction) in the forward projection images. To this end, the forward-projected vessel margins may, for example, be used for an inpainting step. This may be achieved by regular inpainting algorithms, as described by Unberath et al. Alternatively, consistency conditions may also be applied, as described by Preuhs, A. et al. (2020). In the latter case, at least two projection images with the same motion state and at least 20 degrees of projection angle difference (cf. Preuhs, A. et al. (2018)) must be obtained for each image. The two images are then used for iteratively erasing the vascular structures that are not included in the projection by extracting them from a new projection image. The aim of optimization is to maximize the consistency of the newly produced projection images (i.e., of the extracted vessels). Both the standard inpainting method and the consistency-based method lead to a projection image stack in which all the non-feeder-vessels have been removed.

In a fifth step S5, a secondary 3D reconstruction is generated from the forward projection images (without the secondary vessels or non-feeder vessels) with automatic motion compensation. For example, a motion-compensated reconstruction of the projection stack may thus be performed, wherein only the feeder vessels are present as high-contrast structures and the non-feeder vessels are suppressed (for example, painted over). The motion compensation used is, for example, a non-rigid motion compensation scheme using high-contrast structures (cf. Rohkohl, C. et al.).

FIG. 3 depicts one of many tomographic images obtained from the primary 3D reconstruction. The image depicts a tumor 12 and a feeder vessel or target vessel 13. The target vessel 13 had already been identified in the primary 3D reconstruction and optionally labeled. This identification or labeling is adopted in the forward projection. Furthermore, the tomographic image, that corresponds to a plane from the primary 3D reconstruction, depicts portions of many further vessels that are non-feeder-vessels and are here denoted secondary vessels 14. These secondary vessels 14 or secondary vessel portions may be readily eliminated or erased in a forward projection image. The secondary vessel portions may, for example, be easily identified or segmented using an image processing algorithm. Optionally, not only is the respective secondary vessel portion erased but also a safety region around this portion. It may in this way be ensured that any motion artifacts of the secondary vessel are also eliminated.

Erasure may, as mentioned, be performed by inpainting. The secondary vessel region to be erased is, for example, replaced by pixels whose brightness corresponds to that of the pixels in the surroundings of the secondary vessel region.

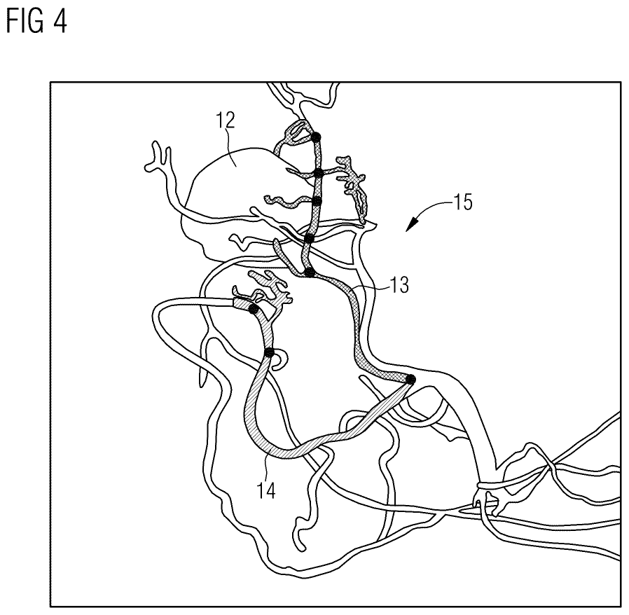

FIG. 4 depicts a forward projection of a secondary 3D reconstruction, that has been generated from the tomographic image according to FIG. 3 and many further tomographic images. It depicts a vascular system 15 in the volume around the tumor 12. This spatial representation depicts a target vessel 13 and a non-feeder vessel (secondary vessel) 14. The target vessel 13 has been identified as a feeder vessel for the tumor 12.

There are thus both feeder and non-feeder vessels. Once the non-feeder vessels have been erased, the motion compensation algorithm may concentrate on the target vessel 13, whereby better motion compensation may typically be achieved. A secondary 3D reconstruction therefore depicts the target vessel 13 as a rule more sharply than does the primary 3D reconstruction. It would thus, for example, be easier for the physician to penetrate the respectively necessary feeder vessel with the catheter in order to embolize the tumor 12.

Advantageously, optimization of non-rigid motion estimation may thus be simplified by simplifying the cost function. Normally, all high-contrast features constitute part of the optimization algorithm. High contrast is defined by a given percentile of the highest intensities in the reconstructed image. In contrast thereto, the high-contrast image features are restricted by removing those vessels that are not involved in the reconstruction. This simplifies the optimization problem and prevents optimization for example from drifting into a local minimum. It is moreover ensured that the clinically relevant features are retained.

It is to be understood that the elements and features recited in the appended claims may be combined in different ways to produce new claims that likewise fall within the scope of the present embodiments. Thus, whereas the dependent claims appended below depend from only a single independent or dependent claim, it is to be understood that these dependent claims may, alternatively, be made to depend in the alternative from any preceding or following claim, whether independent or dependent, and that such new combinations are to be understood as forming a part of the present specification.

While the present embodiments have been described above by reference to various embodiments, it may be understood that many changes and modifications may be made to the described embodiments. It is therefore intended that the foregoing description be regarded as illustrative rather than limiting, and that it be understood that all equivalents and/or combinations of embodiments are intended to be included in this description.

1. A method for motion-compensated reconstruction of a target vessel, the method comprising
providing a primary 3D reconstruction of a volume including the target vessel;
identifying an image of the target vessel and an image of a secondary vessel different from the target vessel in the primary 3D reconstruction;
forward-projecting the image of the target vessel and the image of the secondary vessel onto forward projection images;
erasing the image of the secondary vessel in the forward projection images; and
generating a secondary 3D reconstruction from the forward projection images with automatic motion compensation.

2. The method of claim 1, wherein motion blur of the secondary vessel arises during forward projection, the motion blur being taken into account during erasure with a spatial safety region around the image of the secondary vessel in the forward projection images.

3. The method of claim 1, wherein an inpainting algorithm is used to erase the image of the secondary vessel.

4. The method of claim 1, wherein the image of the secondary vessel is erased in that two projection images in the same motion state but at different projection angles are provided for each of the forward projection images, images of vascular structures of the secondary vessel being extracted from these, and the image of the secondary vessel is erased in the respective forward projection image when the extracted images of the vascular structures and the image of the secondary vessel in the respective projection image fulfill a consistency condition.

5. The method of claim 4, wherein the image of the secondary vessel is erased iteratively in the respective forward projection image by modifying a contrast of the secondary vessel in the projection images in a stepwise manner until the consistency condition is fulfilled.

6. The method of claim 1, wherein non-rigid motion is compensated during the automatic motion compensation.

7. The method of claim 1, wherein the target vessel is an outflow vessel for a given region of an object and the secondary vessel is an inflow vessel for the given region or wherein the target vessel is an inflow vessel for the given region of the object and the secondary vessel is an outflow vessel for the given region.

8. An image processing apparatus for motion-compensated reconstruction of a target vessel, the image processing apparatus comprising:
a storage device for providing a primary 3D reconstruction of a volume including the target vessel; and
a computing device that is configured to:
identify an image of the target vessel and an image of a secondary vessel different from the target vessel in the primary 3D reconstruction,
forward-project the image of the target vessel and the image of the secondary vessel onto forward projection images,
erase the image of the secondary vessel in the forward projection images, and
generate a secondary 3D reconstruction from the forward projection images with automatic motion compensation.

9. A non-transitory computer implemented storage medium, including machine-readable instructions stored therein for motion-compensated reconstruction of a target vessel, the machine-readable instructions when executed by at least one processor, cause the processor to:
provide a primary 3D reconstruction of a volume including the target vessel;
identify an image of the target vessel and an image of a secondary vessel different from the target vessel in the primary 3D reconstruction;
forward-project the image of the target vessel and the image of the secondary vessel onto forward projection images;
erase the image of the secondary vessel in the forward projection images; and
generate a secondary 3D reconstruction from the forward projection images with automatic motion compensation.
